(19) United States
(12) Patent Application Publication
(10) Pub. No.: US 2001/0039404 A1
(43) Pub. Date: Nov. 8, 2001

(54) Cannula

(75) Inventor: Phillipp Rolle, Neumunster (DE)

Correspondence Address: Thomas C. Pontani, Esq., Cohen, Pontani, Lieberman & Pavane, Suite 1210, 551 Fifth Avenue, New York, NY, 10176, US

(73) Assignee: Transcoject Gesellschaft fur medizinische Gerate mbH & Co. KG

(21) Appl. No.: 09/810,382

(22) Filed: Mar. 16, 2001

(30) Foreign Application Priority Data
Mar. 16, 2000 (DE) 100 12 886.6

(51) Int. Cl. A61M 5/00

(52) U.S. Cl. 604/256

(57) ABSTRACT

The cannula is provided for depositing liquid or pasty substances. At the free end of the cannula there is formed a closure body closing this. In the region between the cannula and the closure body there is provided a break-off location so that the closure body by way of a simple breaking may be removed from the cannula. The closure body may then for a later re-closing be placed onto the end of the cannula.

The invention relates to a cannula for depositing liquid or pasty substances.

In order to be able to deposit liquid or pasty substances such as for example dental filling material, adhesives and likewise in a precise and directed manner, cannulas are applied which are placed onto the end of a syringe which serves as a metering vessel. The placing-on of the cannula is effected after the removal of a syringe closure.

Although the application of cannulas for depositing such material has proven itself very well, it is however often the case that not all of the contents of the syringe is required for one application but rather a withdrawal several times is desirable in order to be able to use the whole contents of the syringe bit by bit. Since with these substances at a rule it is the case of substances which harden or dry out, it must be ensured that the syringe after the respective application is sealingly closed again, which means the cannula must be removed and the closure plug must again be placed onto the syringe. Disregarding the fact that the removal of the cannula and the closing of the syringe is relatively time-consuming, with this there also exists the danger that in particular with liquid substances, remains of the substance drip out of the cannula. Moreover for each application a new cannula is required. A cleaning of the cannula is practically not possible since as a rule the inner cross section is dimensioned so small that a cleaning with an economical effort is not possible.

### BRIEF SUMMARY OF THE INVENTION

Against this state of the art it is the object of the invention to design a cannula of the known type such that a repeated application is made possible. Furthermore where possible also the handling of the whole product to which the cannula belongs is to be simplified.

This object is achieved according to the invention by the features specified in claim 1. Advantageous embodiments of the invention are to be deduced from the dependent claims, the subsequent description as well as from the drawing.

The basic concept of the present invention is to render a cleaning of the cannula non-essential given a repeated use, in that this cannula after usage is sealingly closed, thus is not at all removed from the syringe. The closure body itself is formed on the free end of the cannula and firstly closes this (initial condition). There is provided a break-off location in the region between the cannula and the closure body so that the closure body by way of hand force may be separated from the cannula. After the separation of the closure body from the cannula this is open at the end-side (at its distal end). The renewed closing is effected then by way of the closure body which previously has been separated from the cannula.

The invention not only permits cannulas and closure bodies to be designed as an injection molded part of plastic or glass, which is favourable with regard to manufacturing technology, it also yields the advantage that it is always ensured that after the opening there is always present a closure body. Thus it may not occur that a closure body not is packaged with the product. Furthermore the forming of the closure body onto the cannula has the advantage that this in the initial condition is hermetically closed on the end side.

The present invention thus permits also a one-piece design of the syringe body and the cannula which with regard to the manufacturing costs is advantageous.

Alternatively the cannula according to the invention may also as a separate component for example be provided with a Luer connection and/or Luer lock connection so that it may be applied with usual syringes. The cannula may then thus also effect the repeated depositing of substance out of syringes which are known in the state of the art and have been previously described.

The closure body may be designed according to the demands, for example as a closure cap which is put over the free end of the cannula, or also as a plug which is introduced into the free end of the cannula. Also a combination of both closure body forms is conceivable in that within the closure cap additionally there is molded yet a plug so that the free cannula end is surrounded by the closure body on all sides.

It is particularly advantageous when the break-off location lies at the end of the cannula, thus directly in front of the closure body, since then on the one hand the complete cannula length is maintained for depositing and on the other hand the closure body after the removal of a prefilled syringe remains largey free of substance residue.

In order to ensure that the closure body does not inadvertently release from the cannula end, it is useful to provide locking means between the closure body and the cannula. With the use of a closure cap for example within the cap there may be provided a peripheral groove which may be brought into engagement with formations correspondingly provided on the outer periphery of the cannula, or reverse.

It is favorable with regard to manufacturing technology to form the closure body on the end of the cannula such that the closure body for the repeated closing as directed is to be placed on rotated by 180°. Specifically there then results a comparatively simple injection molding tool, and also relatively small lumens may be realised.

In order to simplify the removal of the closure body from the cannula it is useful to form a handle on this. Such a handle enlarges the gripping surface and simplifies the first removal, thus the breaking-off of the closure body from the cannula, as well as the repeated placing on and off, in particular when for example in the edge region between the closure cap and the cannula the substance to be deposited dried or hardens.

### BRIEF DESCRIPTION OF THE DRAWINGS

The invention is hereinafter described in more detail by way of an embodiment example shown in the drawings. There are shown in:

FIGS. 1a-c in a simplified schematic sectional representation, how the cannula according to the invention is to be used as directed,

FIG. 2 in enlarged sectional representation the end of the cannula according to FIG. 1a, with a closure body,

FIG. 3 a second embodiment example of a cannula in a longitudinal section and

FIG. 4 a view in the direction of the arrow IV in FIG. 3.

### DETAILED DESCRIPTION OF THE INVENTION

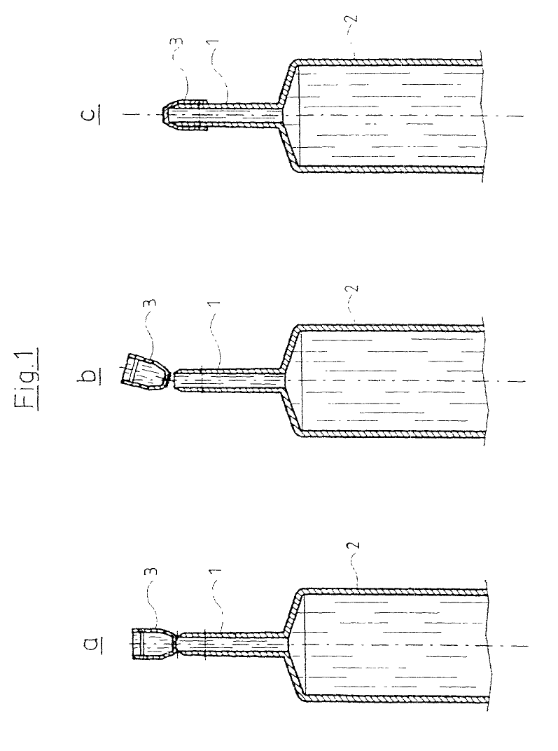

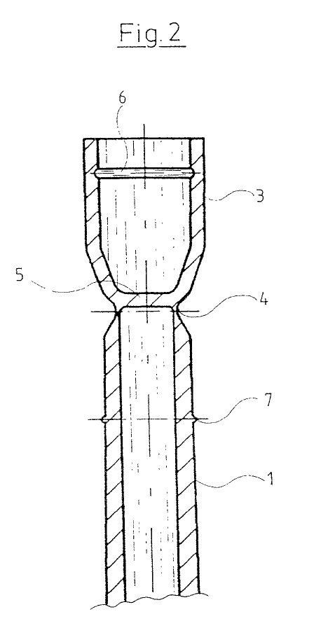

The cannula 1 represented by way of FIGS. 1 and 2 has essentially a cylindrical shape and is formed as one piece with the syringe body into which in a manner known per se there immerses a syringe piston. At the distal end of the cannula 1 there is formed a closure cap 3 which at the same time closes the cannula 1 on the end-side. Between the free end of the cannula 1 and the closure cap 3 there is provided a break-off location 4 which means in this region the wall in comparison to the remaining cannula 1 is formed considerably thinner so that the closure body 3, as indicated in FIG. 3b, may be broken off from the cannula 1 and on the one hand there forms a smooth as possible termination at the cannula end and on the other hand the forces for removing the closure cap are manually mastered. The break-off location 4 is formed such that the cannula towards the end tapers in cross section, whereas the lumen remains constant. This has the advantage that there is formed quasi a tapering tip which simplifies the directed depositing.

The closure cap 3 is formed beaker-shaped and with regard to the inner diameter is dimensioned such that it may be placed inverted onto the cannula 1 after the removal (see FIG. 1c). With this the outer wall of the closure cap 3 runs conically towards the transverse wall 5 in order to ensure a sealed closure in this region. On the inner periphery of the closure cap 3 there is provided a groove 5 which is semicicular in cross section and which is part of a locking connection. Complementarily to this groove 6 there is provided on the outer periphery of the cannula 1 a ring which likewise has a semicircular cross section so that on placing the closure cap 3 onto the end of the cannula 1 (FIG. 1c) this ring 7 engages into the groove 6 by which means the closure cap 3 is reliably held on the cannula end. According to the material used the ring 7 may also be formed by individual projections or there may be provided a different locking connection.

The use of the previously described cannula is effected as follows:

A syringe cylinder filled with the substance to be deposited, e.g. a dental filling mass, is at its open end closed by a syringe piston so that the substance within the syringe is hermetically closed (FIG. 1a). For use then by rotating or bending, the closure cap 3 is separated from the cannula 1 (FIG. 1b). By way of this the cannula at its free end, thus the end distant to the syringe body is opened. By way of pressing in the non-shown syringe piston the substance located in the syringe body is deposited via the cannula 1. The cannula 1 is thereafter again closed by placing on the closure cap 3 in the manner shown in FIG. 1c. By way of the locking connection between the groove 6 and ring 7, the closure cap 3 is seated sealingly and rigidly on the end of the cannula 1. For the renewed depositing then the closure cap 3 is removed and after the depositing is placed on again. The substance located in the syringe body 2 and within the cannula 1 is after placing on the closure cap sealed from the outer atmoshphere so that there does not exist the danger that this hardens or dries out within the cannula. The cannula may thus be used again until the whole syringe contents have been used up. Since with the embodiment according to FIGS. 1 and 2 the syringe body 2, cannula 1 and closure cap 3 are formed as one piece as an injection molded part of plastic, the manufacturing costs are very favorable and in comparison to a simple syringe body—assuming a sufficient piece number—are only insignificantly higher.

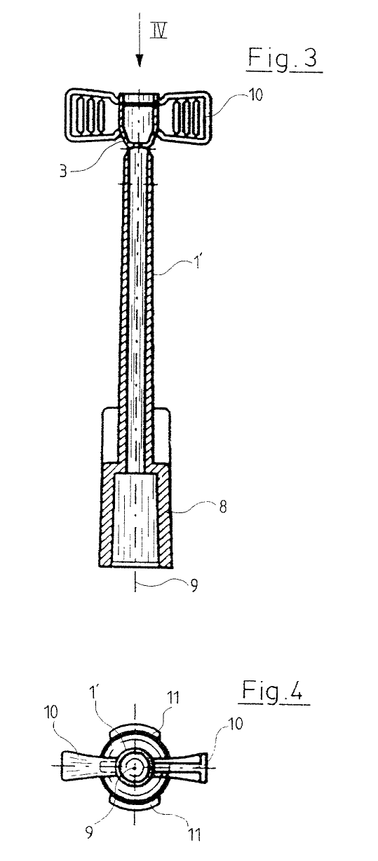

The embodiment according to the FIGS. 3 and 4 differs from that previously described in that the cannula 1 is not formed as one piece with the syringe body 2 but as a separate cannula part, as this is also known from the state of the art. The cannula 1′ is therefore at its syringe-end side provided with a Luer connection 8. Otherwise the design corresponds essentially to that previously described. For the improved handling there are formed onto the closure cap 3 two wings 10 laterally displaced by 180° to one another (with respect to the cannula axes 9), these forming a handle. These wings 10 give a good hold so that the closure cap 3 may be securely held between the fingers and the thumb which is particularly advantageous for the first breaking-off from the cannula 1′ but also with a later placing on and off.

As may be recognised in the plan view according to FIG. 4 the Luer connection 8 in the outer periphery is provided with two projections 11, so that also a connection with a Luer connection is possible as this is known per se with cannulas from medicine technology.

The handling of the cannula 1′ is effected in an analogous manner to the previously described one, with the difference that before the first use the Luer connection is connected to the filled syringe.

### List of Reference Numerals

1.1′—cannula

2—syringe body

3—closure cap

4—break-off location

5—transverse wall

6—groove

1. A cannula for depositing liquid or pasty substances, wherein at the free end of the cannula there is formed a closure body closing this, wherein a break-off location is provided in the region between the cannula and the closure body and wherein the closure body is designed such that it after the separation from the cannula may be applied for the renewed closing of this

2. A cannula according to claim 1, characterized in that the closure body is designed as a cap which after separating from the cannula may be placed on this for the purpose of the renewed closure.

3. A cannula according to claim 1, wherein the closure body is designed plug-like and after the separation from the cannula is insertable into this for the purpose of a renewed closing.

4. A cannula according to one of the preceding claims, wherein if at its syringe-side end is provided a Luer connection or a Luer lock connection.

5. A cannula according to one of the preceding claims, wherein it is formed onto the end of a syringe.

6. A cannula according to one of the preceding claims, wherein a break-off location is provided at the end of the cannula directly in front of the closure body.

7. A cannula according to one of the preceding claims, wherein the locking means are provided between the cannula and closure body

8. A cannula according to one of the preceding claims, wherein the closure body is formed onto the cannula end such that if after separation from the cannula for the renewed closure is to be placed on rotated by 180°

9. A cannula according to one of the preceding claims, wherein on the closure body there is formed on at least one handle
